(19) United States
(12) Patent Application Publication
(10) Pub. No.: US 2022/0305169 A1
(43) Pub. Date: Sep. 29, 2022

(54) DEGRADABLE HEMOSTATIC SPONGE AND PREPARATION METHOD AND USE THEREOF, AND DEGRADABLE DRUG-LOADED HEMOSTATIC SPONGE

(71) Applicant: Hangzhou Vitex Medical Device Co., Ltd, Hangzhou City (CN)

(72) Inventors: Peipei CAO, Hangzhou City (CN); Jonathon Zhong ZHAO, Hangzhou City (CN); Guanya WU, Hangzhou City (CN)

(21) Appl. No.: 17/370,601

(22) Filed: Jul. 8, 2021

(30) Foreign Application Priority Data
Mar. 25, 2021 (CN) 202110319395.1

(51) Int. Cl. A61L 15/42; A61L 15/44; A61L 15/64; A61L 15/28; C08J 9/28

(52) U.S. Cl. CPC A61L 15/425; A61L 15/44; A61L 15/64; A61L 15/28; C08J 9/28; A61L 2300/222; A61L 2300/604; A61L 2300/802; A61L 2300/606; C08J 2301/32; C08J 2303/04; C08J 2201/0484; C08J 2207/10; A61L 2300/40

(57) ABSTRACT

The present disclosure belongs to the technical field of hemostatic materials, and specifically relates to a degradable hemostatic sponge and a preparation method and use thereof, and a degradable drug-loaded hemostatic sponge. The degradable hemostatic sponge provided by the present disclosure is prepared from raw materials including a crosslinking-modified starch and a cellulose through freeze-drying, where a mass ratio of the crosslinking-modified starch to the cellulose is (0.2-5):1. The degradable hemostatic sponge provided by the present disclosure has a high water-absorbing rate and a large water-absorbing capacity, shows a high support strength and a long support time after water absorption, and is made from plant-derived raw materials and thus may be completely biodegraded. The degradable drug-loaded starch hemostatic sponge provided by the present disclosure has a drug-loaded coating attached to a surface of the sponge, where the drug is slowly released while a support is maintained.

### CROSS REFERENCE TO RELATED APPLICATION

This patent application claims the benefit and priority of Chinese Patent Application No. 202110319395.1 filed on Mar. 25, 2021, the disclosure of which is incorporated by reference herein in its entirety as part of the present application.

### TECHNICAL FIELD

The present disclosure belongs to the technical field of hemostatic materials, and specifically relates to a degradable hemostatic sponge and a preparation method and use thereof, and a degradable drug-loaded hemostatic sponge.

### BACKGROUND ART

At present, there are a wide variety of hemostatic materials on the market, which are made with starch and derivatives thereof, chitosan and derivatives thereof, gel, cellulose and derivatives thereof, alginate, and polyurethane as main raw materials, and have been used maturely in clinical practice.

As one of the most common plant polysaccharides, starch is extracted from plants, and has advantages of wide source, renewability, high quality and low price. The sources of starch are safe, and the adverse reactions caused by impurity proteins in animal-derived materials may be avoided. Starch is widely used in food, medicine and other aspects due to excellent biocompatibility. Starch is hydrolyzed into glucose under enzyme catalysis in human body, which may be fully absorbed and metabolized without toxic and side effects. Starch is mainly used to prepare hemostatic powders, among commercially available products. As early as 2000, FDA had approved the Arista hemostatic powder produced by Medaor with starch as a raw material to be used as a hemostatic agent for emergency treatments and clinical operations. There are similar products in China by now, such as the Quickclean product produced by Hangzhou Singclean Medical Products Co., Ltd. and Healsoon product produced by Beijing Aitekang Medical Co., Ltd., which are both hemostatic powders made by potato starch being subject to crosslinking modification with epichlorohydrin.

Chinese patent CN202010225321.7 discloses a hemostatic sponge formed through freeze- drying with starch and glycerin as raw materials. Chinese patent CN201510545987.X discloses a hemostatic sponge formed through freeze-drying with starch, polyethylene glycol and gallic acid as raw materials. However, the hemostatic sponges prepared through freeze-drying disclosed in the above patents have a low water-absorbing rate and a poor support strength after water absorption.

The commercially available Nasopore used after surgery may maintain support for a long time, but after it is degraded, many polyurethane residues will remain, which are non-degradable.

### SUMMARY

In view of this, the present disclosure provides a degradable hemostatic sponge and a preparation method and use thereof, and a degradable drug-loaded hemostatic sponge. The degradable hemostatic sponge provided by the present disclosure has a high water-absorbing rate and a large water-absorbing capacity, and shows a high support strength and a long support time after water absorption. Moreover, the degradable hemostatic sponge is made from plant-derived raw materials and may be completely biodegraded.

The degradable drug-loaded starch hemostatic sponge provided by the present disclosure has a drug-loaded coating attached to a surface of the sponge, where the slow release of the drug is achieved through the slow degradation of an adjuvant in the drug coating, and the adjuvant composition may be adjusted to achieve a drug release time comparable to a sponge support time. After the degradable drug-loaded starch hemostatic sponge is placed at an action site, the drug is slowly released while the support is maintained.

The present disclosure provides a degradable hemostatic sponge, which is prepared from raw materials including a crosslinking-modified starch and a cellulose through freeze-drying;

a mass ratio of the crosslinking-modified starch to the cellulose is (0.2-5):1.

In some embodiments, the cellulose may be one or more selected from the group consisting of methyl cellulose, ethyl cellulose, carboxymethyl cellulose, hydroxyethyl cellulose and hydroxypropyl cellulose;

the cellulose may have a number average molecular weight of 100,000 to 1,500,000;

a crosslinking agent in the crosslinking-modified starch may be one or more selected from the group consisting of epichlorohydrin, formaldehyde, glutaraldehyde, metaphosphate, oxalate and terephthaloyl chloride;

a mass of the crosslinking agent may be 1% to 50% of a mass of the starch.

The present disclosure provides a preparation method of the degradable hemostatic sponge according to the above technical solutions, including the following steps:

mixing the crosslinking-modified starch, the cellulose and water to obtain a freeze-drying solution;

pre-freezing and freeze-drying the freeze-drying solution successively;

where a mass ratio of the crosslinking-modified starch to the cellulose is (0.2-5):1.

In some embodiments, the freeze-drying solution may have a solid-to-liquid ratio of 1:(5-100);

the pre-freezing may be conducted at a temperature of −30° C. to −40° C. and a holding time of 2 h to 4 h;

the pre-freezing may be conducted in a closed mould, and the mould may be made of metal.

In some embodiments, the freeze-drying may be conducted at a temperature of −50° C. to −60° C., a holding time of 48 h to 72 h, and a vacuum degree of −0.1 MPa.

In some embodiments, after the mixing, the preparation method may further include: degassing a mixed solution obtained from the mixing; the degassing may be one or more selected from the group consisting of ultrasonic degassing, standing degassing and centrifugal degassing;

the ultrasonic degassing may be conducted for 10 min to 60 min at an ultrasonic frequency of 30 KHz to 50 KHz and an ultrasonic power of 100 W to 1,500 W;

the standing degassing may be conducted for 12 h to 24 h;

the centrifugal degassing may be conducted for 5 min to 20 min at a centrifugal rotational speed of 3,000 rpm to 8,000 rpm.

The present disclosure provides use of the degradable hemostatic sponge according to the above technical solutions or a degradable hemostatic sponge obtained by the preparation method according to the above technical solutions as a hemostatic material and an anti-adhesion material and in the preparation of a drug slow-release material.

The present disclosure provides a degradable drug-loaded hemostatic sponge, including a degradable hemostatic sponge, and a drug and a pharmaceutical adjuvant that are loaded on a surface of the degradable hemostatic sponge.

In some embodiments, the drug may include glucocorticoid drugs or antihistamine drugs;

the pharmaceutical adjuvant may include one or more of a slow-release agent, an adhesive and a disintegrant.

In some embodiments, the slow-release agent may include one or more of polylactide, polyglycolide, poly(lactide-co-glycolide) copolymer, polycaprolactone and polyhydroxybutyrate-valerate;

the adhesive may include one or more of gelatin, cellulose and polyethylene glycol.

The present disclosure provides a degradable hemostatic sponge, which is prepared from raw materials including a crosslinking-modified starch and cellulose through freeze-drying. A mass ratio of the crosslinking-modified starch to the cellulose is (0.2-5):1. In the present disclosure, due to the viscosity of the cellulose, a formed hemostatic sponge has specified elasticity and toughness; also, a mass ratio of the crosslinking-modified starch to cellulose is adjusted such that a hemostatic sponge formed through freeze-drying may not have strip or sheet shaped crystal patterns, thereby avoiding the poor support strength and poor tensile strength of sponge caused by the strip or sheet shaped crystal patterns. Moreover, in the present disclosure, a crosslinking-modified starch is used as a main raw material and cellulose with a specified viscosity is used as an auxiliary material, both of which are derived from plants and may be enzymatically decomposed into glucose in the human body and finally decomposed into carbon dioxide and water, so that an excellent biocompatibility and safety is achieved. Results in the examples show that the degradable drug-loaded hemostatic sponge prepared from the degradable hemostatic sponge provided by the present disclosure, with a size of 5.0 cm×2.0 cm×1.2 cm, has a water absorption ratio maintained 30 or more and completes water absorption within 20 s, indicating that the degradable drug-loaded hemostatic sponge provided by the present disclosure has the advantages of a high water-absorbing rate, a large water-absorbing capacity and a strong water-locking ability. Furthermore, the degradable drug-loaded hemostatic sponge, when being compressed to a thickness of 4 mm, has a support strength of 10 N to 16 N, indicating that the degradable drug-loaded hemostatic sponge provided by the present disclosure has the advantage of a high support strength.

The degradable drug-loaded starch hemostatic sponge provided by the present disclosure has a drug-loaded coating attached to a surface of the sponge, where the slow release of the drug is achieved through the slow degradation of an adjuvant in the drug coating, and the adjuvant composition may be adjusted to achieve a drug release time comparable to a sponge support time. After the degradable drug-loaded starch hemostatic sponge is placed at an action site, the drug is slowly released while the support is maintained.

### BRIEF DESCRIPTION OF THE DRAWINGS

FIG. 1 is a physical picture of the degradable hemostatic sponges prepared in Example 1; and

FIG. 2 is a diagram showing the change in support strength with compression deformation of the degradable hemostatic sponges prepared in Example 1.

### DETAILED DESCRIPTION OF THE EMBODIMENTS

The present disclosure provides a degradable hemostatic sponge, which is prepared from raw materials including a crosslinking-modified starch and a cellulose through freeze-drying;

a mass ratio of the crosslinking-modified starch to the cellulose is (0.2-5):1.

In the present disclosure, unless otherwise specified, the raw materials used are all commercially available products well known to those skilled in the art.

In the present disclosure, the cellulose may preferably be one or more selected from the group consisting of methyl cellulose, ethyl cellulose, carboxymethyl cellulose, hydroxyethyl cellulose and hydroxypropyl cellulose, and more preferably carboxymethyl cellulose or hydroxyethyl cellulose. In the present disclosure, the cellulose may have a number average molecular weight preferably of 100,000 to 1,500,000, more preferably of 150,000 to 1,200,000, and most preferably of 500,000 to 1,000,000.

In the present disclosure, a crosslinking agent in the crosslinking-modified starch may preferably be one or more selected from the group consisting of epichlorohydrin, formaldehyde, glutaraldehyde, metaphosphate, oxalate and terephthaloyl chloride, where the metaphosphate may be sodium metaphosphate and/or potassium metaphosphate in some embodiments, and the oxalate may be sodium oxalate and/or potassium oxalate in some embodiments. In the present disclosure, the crosslinking agent may more preferably be epichlorohydrin or terephthaloyl chloride. In the present disclosure, the crosslinking agent may be used in the form of a crosslinking agent aqueous solution in some embodiments, and the present disclosure has no specific limitations on a concentration of the crosslinking agent aqueous solution.

In the present disclosure, a mass of the crosslinking agent may be preferably 1% to 50%, more preferably 10% to 45%, and most preferably 15% to 35% of a mass of the starch.

In the present disclosure, the crosslinking agent reacts with the starch via hydroxyl such that two or more starch molecules are crosslinked to form a crosslinking-modified starch polymer with a spatial network structure. The crosslinking-modified starch has shear resistance and acid resistance.

In the present disclosure, in some embodiments, a preparation method of the crosslinking-modified starch may include the following steps:

mixing starch and water for gelatinization to obtain a gelatinized starch solution;

mixing the gelatinized starch solution with a crosslinking agent for a crosslinking reaction to obtain the crosslinking-modified starch.

In the present disclosure, starch is mixed with water for gelatinization to obtain a gelatinized starch solution. In the present disclosure, the starch may preferably be one or more selected from the group consisting of potato starch, tapioca starch, corn starch, sweet potato starch and wheat starch, and may more preferably be potato starch or corn starch. In the present disclosure, the starch solution obtained by mixing the starch and water may have a mass concentration preferably of 0.5% to 20%, more preferably of 1.5% to 15%, and most preferably of 2.5% to 12%. The present disclosure has no specific limitations on a specific implementation process for mixing the starch and water. In a specific example of the present disclosure, the mixing is conducted in a reactor.

In the present disclosure, the gelatinization may be conducted at a temperature preferably of 50° C. to 80° C., and more preferably of 60° C. to 75° C.; the gelatinization may be conducted preferably for 0.5 h to 2 h, and more preferably for 1 h to 1.5 h. In the present disclosure, the gelatinization may be conducted under stirring in some embodiments, and the stirring may be conducted at a speed preferably of 150 rpm to 350 rpm and more preferably of 200 rpm to 250 rpm. The present disclosure has no specific limitations on a specific implementation of the stirring. In a specific example of the present disclosure, the stirring is conducted in a reactor.

In the present disclosure, the gelatinized starch solution may have a pH of 9 to 10 in some embodiments; the pH may be adjusted by a pH adjusting agent in some embodiments; the pH adjusting agent may be a sodium hydroxide solution in some embodiments; the sodium hydroxide solution may have a concentration of 1 mol/L in some embodiments.

In the present disclosure, after a gelatinized starch solution is obtained, the gelatinized starch solution is mixed with a crosslinking agent for a crosslinking reaction to obtain a crosslinking-modified starch.

In the present disclosure, the mixing of the gelatinized starch solution and the crosslinking agent may be conducted by adding the crosslinking agent aqueous solution according to the above technical solutions dropwise into the gelatinized starch solution in some embodiments, and the dropping speed may be 1 drop/1 s in some embodiments. In the present disclosure, the gelatinized starch solution and the crosslinking agent may be mixed at a temperature preferably of 45° C. to 60° C., and more preferably of 50° C. to 55° C. In the present disclosure, the mixing may be conducted under stirring in some embodiments, and the stirring may be conducted at the same speed as the stirring for the gelatinization in some embodiments.

In the present disclosure, the crosslinking reaction may be conducted at a temperature the same as that for the mixing of the gelatinized starch solution and the crosslinking agent in some embodiments, and the crosslinking reaction may be conducted preferably for 2 h to 6 h and more preferably for 3 h to 5 h. In the present disclosure, the crosslinking reaction may be conducted under stirring in some embodiments, and the stirring may be conducted at the same speed as the stirring for the gelatinization in some embodiments.

In the present disclosure, in some embodiments, after the crosslinking reaction is completed, a crosslinking reaction solution may be subjected to a post-treatment to obtain the crosslinking- modified starch. In the present disclosure, in some embodiments, the post-treatment may include: solid precipitation, solid-liquid separation, washing and drying that are conducted successively. In the present disclosure, a solvent used for the solid precipitation may be ethanol in some embodiments, and a volume ratio of the solvent for the solid precipitation to the reaction system solution may be preferably (1-10):1 and more preferably (2-5):1. The present disclosure has no specific limitations on a specific implementation process of the solid-liquid separation. In the present disclosure, in some embodiments, after a solid product is obtained, the solid product may be washed. In the present disclosure, a solvent used for the washing may be ethanol in some embodiments, the ethanol may be used at a volume the same as that of the solvent for the solid precipitation in some embodiments, and the washing may be conducted for 2 to 3 times in some embodiments. In the present disclosure, in some embodiments, a washed solid product may be dried. In the present disclosure, the drying may be conducted at 50° C. to 60° C. in some embodiments, and the drying may be conducted for 24 h to 30 h in some embodiments.

In the present disclosure, a mass ratio of the crosslinking-modified starch to the cellulose may be (0.2-5):1, preferably (1-4):1, and more preferably (1.5-3):1. The present disclosure has no specific limitations on a specific implementation process of the freeze-drying.

In the present disclosure, due to the viscosity of the cellulose, a formed hemostatic sponge has specified elasticity and toughness; also, a mass ratio of the crosslinking-modified starch to the cellulose is adjusted such that a hemostatic sponge formed through freeze-drying may not have strip or sheet shaped crystal patterns, thereby avoiding the poor support strength, poor tensile strength and low water-absorbing rate of the sponge caused by the strip or sheet shaped crystal patterns. Moreover, in the present disclosure, a crosslinking-modified starch is used as a main raw material and cellulose with a specified viscosity is used as an auxiliary material, both of which are derived from plants and may be enzymatically decomposed into glucose in the human body and finally decomposed into carbon dioxide and water, so an excellent biocompatibility and safety is achieved.

The present disclosure provides a preparation method of the degradable hemostatic sponge according to the above technical solutions, including the following steps:

mixing the crosslinking-modified starch, the cellulose and water to obtain a freeze-drying solution;

pre-freezing and freeze-drying the freeze-drying solution successively;

where a mass ratio of the crosslinking-modified starch to the cellulose is (0.2-5):1.

In the present disclosure, the crosslinking-modified starch, the cellulose and water are mixed to obtain a freeze-drying solution.

In the present disclosure, in some embodiments, the mixing of the crosslinking-modified starch, the cellulose and water may include the following steps:

subjecting the crosslinking-modified starch and part of the water to a first mixing to obtain a crosslinking-modified starch solution;

subjecting the cellulose and the remaining water to a second mixing to obtain a cellulose solution;

subjecting the crosslinking-modified starch solution and the cellulose solution to a third mixing.

In the present disclosure, the crosslinking-modified starch and part of the water are subjected to a first mixing to obtain a crosslinking-modified starch solution. In the present disclosure, the crosslinking-modified starch solution may have a concentration of 0.03 g/mL to 0.05 g/mL in some embodiments; the first mixing may be conducted at room temperature in some embodiments; the first mixing may be conducted for 20 min to 30 min in some embodiments. The first mixing may be conducted under stirring in some embodiments, and the present disclosure has no specific limitations on a specific implementation process of the stirring.

In the present disclosure, the cellulose and the remaining water are subjected to a second mixing to obtain a cellulose solution. In the present disclosure, the cellulose may have a mass content of 0.5% to 6% in the cellulose solution. The second mixing may be conducted at 30° C. to 50° C. in some embodiments, and the second mixing may be conducted for 2 h to 4 h in some embodiments. In the present disclosure, after the second mixing is completed, a pH of the cellulose solution may be adjusted to 3 to 4 in some embodiments, where a pH adjusting agent used may be hydrochloric acid in some embodiments, and the hydrochloric acid may have a concentration of 1 mol/L in some embodiments. The second mixing may be conducted under stirring in some embodiments, and the stirring may be conducted at a speed of 300 rpm to 500 rpm in some embodiments. The present disclosure has no specific limitations on a specific implementation process of the stirring.

In the present disclosure, after a crosslinking-modified starch solution and a cellulose solution are obtained, the crosslinking-modified starch solution and the cellulose solution are subjected to a third mixing to obtain a mixed solution. In the present disclosure, the third mixing may be conducted at room temperature in some embodiments, and the third mixing may be conducted for 30 min to 50 min in some embodiments. The third mixing may be conducted under stirring in some embodiments, and the stirring may be conducted at the same speed as that for the second mixing in some embodiments. The present disclosure has no specific limitations on a specific implementation process of the stirring.

In the present disclosure, the freeze-drying solution may have a pH of 5 to 6 in some embodiments, a pH adjusting agent used may be hydrochloric acid in some embodiments, and the hydrochloric acid may have a concentration of 1 mol/L in some embodiments.

In the present disclosure, after a pH of the mixed solution is adjusted, in some embodiments, the mixed solution may be degassed to obtain the freeze-drying solution. In the present disclosure, the degassing may be one or more selected from the group consisting of ultrasonic degassing, standing degassing and centrifugal degassing in some embodiments. The ultrasonic degassing may be conducted for 10 min to 60 min in some embodiments, the ultrasonic frequency may be 30 KHz to 50 KHz in some embodiments, and the ultrasonic power may be 100 W to 1,500 W in some embodiments; the standing degassing may be conducted for 12 h to 24 h in some embodiments; the centrifugal degassing may be conducted for 5 min to 20 min in some embodiments, and the centrifugal rotational speed may be 3,000 rpm to 8,000 rpm in some embodiments. The present disclosure has no specific limitations on a specific implementation process of the ultrasonic degassing, the standing degassing and the centrifugal degassing. In the present disclosure, when the degassing may be two selected from the group consisting of ultrasonic degassing, standing degassing and centrifugal degassing in some embodiments, the degassing may specifically include: conducting ultrasonic degassing and standing degassing successively, conducting centrifugal degassing and standing degassing successively, or conducting centrifugal degassing and ultrasonic degassing successively. In the present disclosure, when the degassing may be three selected from the group consisting of ultrasonic degassing, standing degassing and centrifugal degassing in some embodiments, the degassing may specifically include: conducting centrifugal degassing, ultrasonic degassing and standing degassing successively.

In the present disclosure, after a freeze-drying solution is obtained, the freeze-drying solution is subjected to pre-freezing and freeze-drying successively. In the present disclosure, the pre-freezing may be conducted at a temperature of −30° C. to −40° C. in some embodiments, and may be conducted for a holding time of 2 h to 4 h in some embodiments. The pre-freezing may be conducted in a closed mould in some embodiments. In a specific example of the present disclosure, the closed mould may preferably be a mould with a cover. The pre-freezing mould may preferably be made of metal, and more preferably of copper, stainless steel, aluminum or aluminum alloy.

In the present disclosure, the freeze-drying may be conducted at a temperature of −50° C. to −60° C. in some embodiments, for a holding time of 48 h to 72 h in some embodiments, and at a vacuum degree of −0.1 MPa in some embodiments. In the present disclosure, the freeze-drying may be conducted in an open mould in some embodiments. In a specific example of the present disclosure, after the pre-freezing is completed, the cover of the mould with a cover used in the pre-freezing is preferably removed and then the freeze-drying is conducted.

In the present disclosure, a closed mould is used for the pre-freezing before freeze-drying, and a metal material with a high conductivity coefficient is chosen to make the mould, such that a rapid cooling molding in a short time may be achieved, and also the closed space allows a better molding of sponge with an uniform internal texture, an uniform porosity and a smooth and uniform appearance, thereby improving both the water-absorbing capacity and the water-locking ability of the degradable hemostatic sponge.

The present disclosure provides use of the degradable hemostatic sponge according to the above technical solutions or a degradable hemostatic sponge obtained by the preparation method according to the above technical solutions as a hemostatic material and an anti-adhesion material and in the preparation of a drug slow-release material.

The present disclosure provides a degradable drug-loaded hemostatic sponge, including a degradable hemostatic sponge, and a drug and a pharmaceutical adjuvant that are loaded on a surface of and inside the degradable hemostatic sponge.

In the present disclosure, in some embodiments, the drug may include one or more selected from the group consisting of glucocorticoid drugs, macrolide drugs, antibacterial drugs, antihistamine drugs and antileukotriene drugs, where the glucocorticoid drugs may include budesonide, mometasone furoate or dexamethasone in some embodiments; the macrolide drugs may include an erythromycin-derived drug, tacrolimus or fidaxomicin in some embodiments; the antibacterial drugs may include amoxicillin, cefuroxime axetil, clavulanic acid or levofloxacin in some embodiments; the antihistamine drugs may include diphenhydramine, cetirizine, ranitidine or montelukast in some embodiments; the antileukotriene drugs may include diphenhydramine, cetirizine, ranitidine or montelukast in some embodiments. In a specific example of the present disclosure, the degradable hemostatic sponge has a size of 5.0 cm×2.0 cm×1.2 cm (length×width×height), and each degradable hemostatic sponge has a drug-loading amount preferably of 50 μg to 2,000 μg. In the present disclosure, the drug-loading amount of the degradable hemostatic sponge may be adjusted according to actual needs.

In the present disclosure, in some embodiments, the pharmaceutical adjuvant may include one or more selected from the group consisting of a slow-release agent, an adhesive and a disintegrant. The slow-release agent may preferably include one or more selected from the group consisting of polylactide, polyglycolide, poly(lactide-co-glycolide) copolymer, polycaprolactone and polyhydroxybutyrate-valerate, and may more preferably include polylactide, polyglycolide, poly(lactide-co-glycolide) copolymer, polycaprolactone or polyhydroxybutyrate-valerate. The adhesive may preferably include one or more selected from the group consisting of gelatin, cellulose and polyethylene glycol, and may more preferably include gelatin, cellulose or polyethylene glycol. The present disclosure has no specific limitations on a type of the disintegrant, which may be selected conventionally according to a type of a drug for preparing the degradable drug-loaded hemostatic sponge. In the present disclosure, the pharmaceutical adjuvant is added to improve the attaching ability of the drug coating and the slow-release ability of the drug and assist the absorption of the drug.

In the present disclosure, a mass ratio of the drug to the pharmaceutical adjuvant may be 1:(3-5) in some embodiments. In the present disclosure, when the drug is budesonide or mometasone furoate, the slow-release agent is a mixture of polylactide copolymer with polyethylene glycol or a mixture of poly(lactide-co-glycolide) copolymer with polyethylene glycol. The present disclosure has no specific limitations on a mass ratio of poly(lactide-co-glycolide) copolymer to polyethylene glycol in the mixture.

The degradable drug-loaded starch hemostatic sponge provided by the present disclosure has a drug-loaded coating attached to a surface of the sponge, where the slow release of the drug is achieved through the slow degradation of the adjuvant in the drug coating, and the adjuvant composition may be adjusted to achieve a drug release time comparable to a sponge support time. After the degradable drug-loaded starch hemostatic sponge is placed at an action site, the drug is slowly released while the support is maintained.

The present disclosure provides a preparation method of the degradable drug-loaded hemostatic sponge, including the following steps:

mixing a drug, a pharmaceutical adjuvant and a polar organic solvent to obtain a drug coating solution;

coating the drug coating solution on a surface of and inside the degradable hemostatic sponge.

In the present disclosure, the drug, pharmaceutical adjuvant and polar organic solvent are mixed to obtain a drug coating solution. In the present disclosure, the polar organic solvent may be acetonitrile in some embodiments; the drug, pharmaceutical adjuvant and polar organic solvent may have a mass ratio of 1:3:96 in some embodiments. The present disclosure has no specific limitations on a specific implementation process of the mixing.

In the present disclosure, after a drug coating solution is obtained, the drug coating solution is coated on a surface of the degradable hemostatic sponge. In the present disclosure, the coating may be spray-coating in some embodiments, where the spray-coating may be conducted at a flow rate of 1.5 ml/min in some embodiments, and the spray-coating may be conducted with a spray-coating machine in some embodiments.

In the present disclosure, after the spray-coating is completed, in some embodiments, the degradable hemostatic sponge spray-coated with the drug coating solution may be subjected to a post-treatment to obtain the degradable drug-loaded hemostatic sponge. In the present disclosure, in some embodiments, the post-treatment may include: drying, packaging and sterilization that are conducted successively. In the present disclosure, the drying may be vacuum drying in some embodiments. The present disclosure has no specific limitations on a specific implementation process of the vacuum drying, provided that the organic solvent may be completely removed. The present disclosure has no specific limitations on a specific implementation process of the packaging. In the present disclosure, the sterilization may be irradiation sterilization or ethylene oxide (EO) sterilization in some embodiments. In the present disclosure, an irradiation ray for the irradiation sterilization may preferably be an X-ray, a γ-ray or an electron beam, and may more preferably be cobalt 60 or an electron beam; the irradiation may be conducted at a dosage preferably of 15 kGy to 30 kGy, and more preferably of 20 kGy to 25 kGy.

In order to further illustrate the present disclosure, the technical solutions provided by the present disclosure are described in detail below in connection with examples, but these examples should not be understood as limiting the claimed scope of the present disclosure.

### Example 1

100.0 g of potato starch was weighed and added to a reactor, 4,900 ml of water was added to the reactor, and a resulting mixture was stirred at 200 rpm for 1 h after a temperature of the jacketed reactor was set to 80° C. The temperature of the jacketed reactor was decreased to 50° C., and a pH of a resulting reaction solution was adjusted to 9 with a 1 mol/L NaOH solution. 10 ml of an epichlorohydrin aqueous solution was added dropwise to the reaction solution using a constant-pressure titration funnel, followed by reacting at a constant temperature for 4 h. After the reaction was completed, a resulting reaction solution was poured out and cooled. 12.5 L of absolute ethanol was used for precipitation to obtain a product, which was washed with 2 L of absolute ethanol for 2 to 3 times, and then dried at 50° C. for 24 h to obtain a crosslinking-modified potato starch.

1.0 g of carboxymethyl cellulose was weighed and added to 39 ml of water, and a resulting mixture was stirred at 400 rpm in a 40° C. water bath for 4 h, such that the carboxymethyl cellulose was completely dissolved to obtain a uniform transparent gel. A pH of the carboxymethyl cellulose solution was adjusted to 3 with 1 mol/L HCl, and was cooled to room temperature for later use.

2.0 g of the crosslinking-modified potato starch was weighed and added to 58 ml of water, and a resulting mixture was stirred at room temperature for 20 min, such that the crosslinking-modified potato starch was water-saturated. A resulting solution was poured into the carboxymethyl cellulose solution, and was stirred at 400 rpm for 30 min for a thorough mixing. A pH of a resulting mixed solution was adjusted to 6 with 1 mol/L HCl, and then was stood for 12 h or more to remove bubbles in the solution.

The mixed solution was poured into a pre-freezing mould and stood for 30 min, and then was covered with a mould cover and pre-frozen for 4 h in a −40° C. freezer. After the pre-freezing was completed, the mould cover was removed, and the sample in the mould was freeze-dried at −60° C. and −0.1 MPa for 72 h in a freeze-drying oven to obtain a degradable starch hemostatic sponge.

### Application Example 1

Budesonide, polylactide and acetonitrile were mixed at a mass ratio of 1:3:96 to prepare a drug coating solution. The degradable starch hemostatic sponge was placed and fixed on a tray of a spray-coating machine, and the drug coating solution was spray-coated on the starch hemostatic sponge with the drug spray-coating machine at a flow rate of 0.15 mL/min, where the budesonide had a loading amount of 200 μg. After the spray-coating was completed, the starch hemostatic sponge was vacuum-dried at room temperature for 24 h, packaged and sealed, and sterilized at an irradiation dosage of 25 kGy to obtain a degradable drug-loaded starch hemostatic sponge.

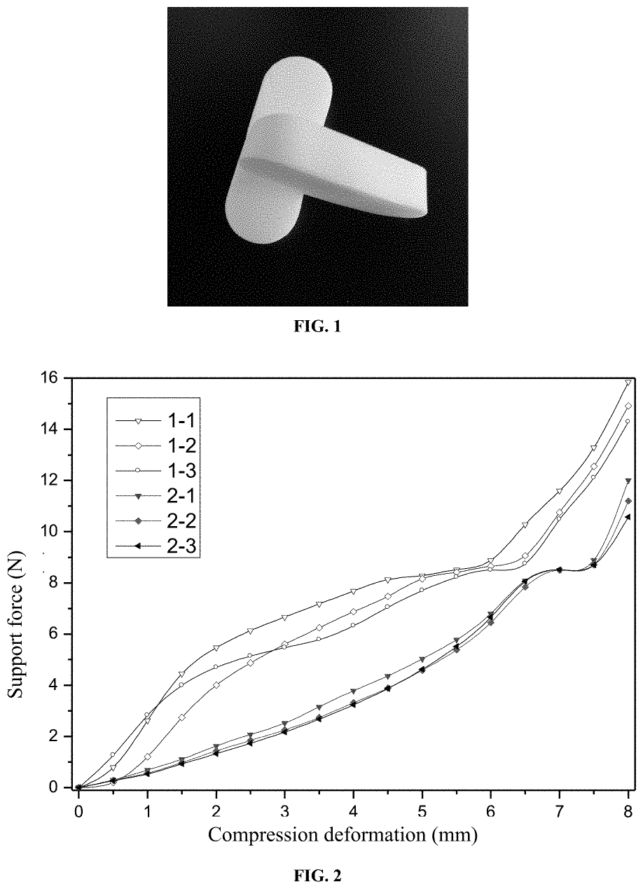

FIG. 1 is a physical picture of the degradable drug-loaded starch hemostatic sponge prepared in this application example. It may be seen from FIG. 1 that the degradable drug-loaded starch hemostatic sponge prepared in this example has a smooth and uniform appearance without surface bulges.

### Example 2

The crosslinking-modified potato starch was prepared by the same method as in Example 1.

0.5 g of hydroxyethyl cellulose was weighed and added to 29.5 ml of purified water, and a resulting mixture was stirred at 400 rpm in a 50° C. water bath for 2 h, such that the hydroxyethyl cellulose was completely dissolved to obtain a uniform transparent gel. A pH of the hydroxyethyl cellulose solution was adjusted to 3 with 1 mol/L HCl, and was cooled to room temperature for later use.

2.5 g of the crosslinking-modified potato starch was weighed and added to 67.5 ml of purified water. After being water-saturated under stirring, a resulting solution was poured into the hydroxyethyl cellulose solution, and was stirred at 400 rpm for 30 min for a thorough mixing. A pH of a resulting mixed solution was adjusted to 6 with 1 mol/L HCl. Then the mixed solution was placed in a centrifuge tube and centrifuged at a centrifugal rotational speed of 5,000 rpm for 10 min to remove bubbles in the solution.

### Application Example 2

The degradable hemostatic sponge prepared in Example 2 was used to prepare a degradable drug-loaded hemostatic sponge by the same preparation method as in Application Example 1.

### Test Example 1

3 sponges of Example 1 and 3 sponges of Example 2 with a size of 5.0 cm×2.0 cm×1.2 cm (length×width×height) were taken, accurately weighed (an obtained mass was recorded as m), and placed in purified water, respectively. The timer was started. A time required for a sample to be water-saturated was recorded, and the water-saturated sample was weighed (an obtained mass was recorded as M). A water absorption ratio was calculated according to the following formula: water absorption ratio=(M−m)/m. Results were shown in Table 1.

TABLE 1
Test data of water-absorbing rate and water-absorbing
capacity of the degradable starch hemostatic sponges

| | | Dry | | Water | Water |
|---|---|---|---|---|---|
| Sample | Preparation | mass | Mass after water | absorption | absorption |
| No. | basis | m/g | absorption M/g | ratio | time/s |
| 1-1 | Example 1 | 0.29 | 10.52 | 36.28 | 8 |
| 1-2 | Example 1 | 0.30 | 10.35 | 34.50 | 12 |
| 1-3 | Example 1 | 0.30 | 10.21 | 34.03 | 10 |
| 2-1 | Example 2 | 0.30 | 11.50 | 38.33 | 15 |
| 2-2 | Example 2 | 0.31 | 10.95 | 35.32 | 17 |
| 2-3 | Example 2 | 0.31 | 11.08 | 35.74 | 14 |

According to the test results of the samples of Examples 1 and 2 in Table 1, it may be seen that all the samples have water absorption ratios maintained at 30 or more and complete water absorption within 20 s, indicating that the degradable drug-loaded hemostatic sponge provided by the present disclosure has the advantages of a high water-absorbing rate, a large water-absorbing capacity and a strong water-locking ability.

### Test Example 2

3 sponges of Example 1 and 3 sponges of Example 2 with a size of 5.0 cm×2.0 cm×1.2 cm (length×width×height) were taken, respectively, and a universal tensile tester was used to test the degradable drug-loaded starch sponges for the tensile strength. During the test, two ends of a sponge were clamped, and the sponge was compressed downward from an initial height to achieve a deformation of 8 mm. The downward compression during the test was conducted at a speed of 20 mm/s. A sensor with a model of BSA-25 kg (M8 3.0 mV/V) produced by Transcell Technology Inc was used for the test. Test results of the change in support strength with compression deformation were shown in Table 2 and FIG. 2. It may be seen from FIG. 2 that the degradable drug-loaded starch hemostatic sponge prepared in Example 1, when compressed to a thickness of 4 mm, has a support strength of 14 N to 16 N, and the degradable drug-loaded starch hemostatic sponge prepared in Example 2, when compressed to a thickness of 4 mm, has a support strength of 10 N to 12 N, indicating that the degradable drug-loaded hemostatic sponge provided by the present disclosure has the advantage of a high support strength.

TABLE 2
Change in support strength with compression deformation
of the degradable starch hemostatic sponges

| Compres- | Example 1 | | | Example 2 | | |
|---|---|---|---|---|---|---|
| sion defor- | Support strength/N | | | Support strength/N | | |
| mation/mm | 1-1 | 1-2 | 1-3 | 2-1 | 2-2 | 2-3 |
| 0 | 0 | 0 | 0 | 0 | 0 | 0 |
| 0.5 | 0.793 | 0.205 | 1.269 | 0.299 | 0.289 | 0.270 |
| 1.0 | 2.626 | 1.209 | 2.824 | 0.687 | 0.575 | 0.534 |
| 1.5 | 4.451 | 2.745 | 4.001 | 1.112 | 0.984 | 0.922 |
| 2.0 | 5.470 | 4.005 | 4.698 | 1.63 | 1.437 | 1.328 |
| 2.5 | 6.132 | 4.873 | 5.14 | 2.076 | 1.846 | 1.737 |
| 3.0 | 6.671 | 5.601 | 5.474 | 2.52 | 2.258 | 2.171 |
| 3.5 | 7.181 | 6.246 | 5.779 | 3.162 | 2.743 | 2.677 |
| 4.0 | 7.684 | 6.873 | 6.329 | 3.787 | 3.321 | 3.232 |
| 4.5 | 8.135 | 7.473 | 7.047 | 4.366 | 3.889 | 3.875 |
| 5.0 | 8.282 | 8.159 | 7.700 | 5.029 | 4.581 | 4.621 |
| 5.5 | 8.519 | 8.420 | 8.221 | 5.784 | 5.385 | 5.518 |
| 6.0 | 8.883 | 8.651 | 8.499 | 6.802 | 6.450 | 6.660 |
| 6.5 | 10.274 | 9.061 | 8.75 | 8.090 | 7.835 | 8.059 |
| 7.0 | 11.596 | 10.757 | 10.477 | 8.510 | 8.501 | 8.511 |
| 7.5 | 13.295 | 12.554 | 12.108 | 8.887 | 8.715 | 8.698 |
| 8.0 | 15.844 | 14.919 | 14.291 | 12.010 | 11.200 | 10.574 |

Although the present disclosure has been described in detail through the above examples, these examples are only a part rather than all of the examples of the present disclosure. All other examples obtained by a person based on these examples without creative efforts shall fall within a protection scope of the present disclosure.

1.-10. (canceled)

11. A degradable hemostatic sponge, wherein the degradable hemostatic sponge is prepared from raw materials comprising a crosslinking-modified starch and a cellulose through freeze-drying;
a mass ratio of the crosslinking-modified starch to the cellulose is (0.2-5):1.

12. The degradable hemostatic sponge according to claim 11, wherein the cellulose is one or more selected from the group consisting of methyl cellulose, ethyl cellulose, carboxymethyl cellulose, hydroxyethyl cellulose and hydroxypropyl cellulose;
the cellulose has a number average molecular weight of 100,000 to 1,500,000;
a crosslinking agent in the crosslinking-modified starch is one or more selected from the group consisting of epichlorohydrin, formaldehyde, glutaraldehyde, metaphosphate, oxalate and terephthaloyl chloride;
a mass of the crosslinking agent is 1% to 50% of a mass of the starch.

13. A preparation method of the degradable hemostatic sponge according to claim 11, comprising the following steps:
mixing the crosslinking-modified starch, the cellulose and water to obtain a freeze-drying solution;
pre-freezing and freeze-drying the freeze-drying solution successively;
wherein a mass ratio of the crosslinking-modified starch to the cellulose is (0.2-5):1.

14. The preparation method according to claim 13, wherein the freeze-drying solution has a solid-to-liquid ratio of 1:(5-100);
the pre-freezing is conducted at a temperature of −30° C. to −40° C. and a holding time of 2 h to 4 h;
the pre-freezing is conducted in a closed mould, and the mould is made of metal.

15. The preparation method according to claim 13, wherein the freeze-drying is conducted at a temperature of −50° C. to −60° C., a holding time of 48 h to 72 h, and a vacuum degree of −0.1 MPa.

16. The preparation method according to claim 13, wherein after the mixing, the preparation method further comprises: degassing a mixed solution obtained from the mixing;
the degassing is one or more selected from the group consisting of ultrasonic degassing, standing degassing and centrifugal degassing;
the ultrasonic degassing is conducted for 10 min to 60 min at an ultrasonic frequency of 30 KHz to 50 KHz and an ultrasonic power of 100 W to 1,500 W;
the standing degassing is conducted for 12 h to 24 h;
the centrifugal degassing is conducted for 5 min to 20 min at a centrifugal rotational speed of 3,000 rpm to 8,000 rpm.

17. A degradable drug-loaded hemostatic sponge, comprising a degradable hemostatic sponge, and a drug and a pharmaceutical adjuvant that are loaded on a surface of the degradable hemostatic sponge.

18. The degradable drug-loaded hemostatic sponge according to claim 17, wherein the drug comprises glucocorticoid drugs or antihistamine drugs;
the pharmaceutical adjuvant comprises one or more of a slow-release agent, an adhesive and a disintegrant.

19. The degradable drug-loaded hemostatic sponge according to claim 17 wherein the slow-release agent comprises one or more of polylactide, polyglycolide, poly(lactide-co-glycolide) copolymer, polycaprolactone and polyhydroxybutyrate-valerate;
the adhesive comprises one or more of gelatin, cellulose and polyethylene glycol.

20. The preparation method according to claim 13, wherein the cellulose is one or more selected from the group consisting of methyl cellulose, ethyl cellulose, carboxymethyl cellulose, hydroxyethyl cellulose and hydroxypropyl cellulose;
the cellulose has a number average molecular weight of 100,000 to 1,500,000;
a crosslinking agent in the crosslinking-modified starch is one or more selected from the group consisting of epichlorohydrin, formaldehyde, glutaraldehyde, metaphosphate, oxalate and terephthaloyl chloride;
a mass of the crosslinking agent is 1% to 50% of a mass of the starch.

21. The degradable drug-loaded hemostatic sponge according to claim 18, wherein the slow-release agent comprises one or more of polylactide, polyglycolide, poly(lactide-co-glycolide) copolymer, polycaprolactone and polyhydroxybutyrate-valerate;
the adhesive comprises one or more of gelatin, cellulose and polyethylene glycol.
